(19) United States
(12) Patent Application Publication
(10) Pub. No.: US 2024/0033527 A1
(43) Pub. Date: Feb. 1, 2024

(54) PARAMETER INDICATION FOR ELECTRICAL STIMULATION

(71) Applicant: Medtronic, Inc., Minneapolis, MN (US)

(72) Inventor: Andrew L. Schmeling, Holmen, WI (US)

(21) Appl. No.: 18/361,660

(22) Filed: Jul. 28, 2023

Related U.S. Application Data
(60) Provisional application No. 63/369,723, filed on Jul. 28, 2022.

(51) Int. Cl. A61N 1/36; A61B 5/388

(52) U.S. Cl. CPC A61N 1/36146; A61N 1/3614; A61B 5/388; A61N 1/37247

(57) ABSTRACT

An example method includes obtaining, by processing circuitry, at least one first electrical stimulation parameter of an electrical stimulation program that defines an electrical stimulation therapy deliverable to a patient and determining an electrical characteristic of a stimulation system configured to deliver the electrical stimulation therapy according to the electrical stimulation program. The method further includes determining, based on the electrical characteristic and the at least one first electrical stimulation parameter, a maximum selectable value for a second electrical stimulation parameter of the electrical stimulation program and outputting, for display by a user interface, the maximum selectable value.

This application claims the benefit of U.S. Provisional Patent Application 63/369,723, which was filed on Jul. 28, 2022, and is entitled, “PARAMETER INDICATION FOR ELECTRICAL STIMULATION,” the entire content of which is incorporated herein by reference.

### TECHNICAL FIELD

This disclosure generally relates to electrical stimulation, and more specifically, control of electrical stimulation.

### BACKGROUND

Medical devices may be external or implanted and may be used to deliver electrical stimulation to patients via various tissue sites to treat a variety of symptoms or conditions such as chronic pain, tremor, Parkinson's disease, epilepsy, urinary or fecal incontinence, sexual dysfunction, obesity, or gastroparesis. A medical device may deliver electrical stimulation therapy via one or more leads that include electrodes located proximate to target locations associated with the brain, the spinal cord, pelvic nerves, peripheral nerves, or the gastrointestinal tract of a patient. Stimulation proximate the spinal cord, proximate the sacral nerve, within the brain, and proximate peripheral nerves are often referred to as spinal cord stimulation (SCS), sacral neuromodulation (SNM), deep brain stimulation (DBS), and peripheral nerve stimulation (PNS), respectively. Electrical stimulation may be delivered by the medical device as a train of pulses, and the values of the parameters defining the pulses may be altered.

### SUMMARY

In general, systems, devices, and techniques are described for managing the delivery of electrical stimulation based on certain factors, which include user input and system capabilities. A stimulation system may deliver different pulse trains defined by one or more different parameter values and/or delivered via different electrode combinations. Although these different pulse trains may deliver different stimulation pulses to the patient, the different pulse trains may be linked in how they are perceived by the patient or how they elicit a perceived effect for the patient. The patient or other user, such as a clinician, may desire to change a parameter and resulting stimulation effects, such as increase or decrease the amplitude of stimulation. The ability of the system to output a pulse train according to parameters including a user-selected parameters may depend on several factors including available power (e.g., available battery voltage and/or electrical current amplitude), pulse frequency, pulse width, the number of cathodes and anodes that can be selected to deliver the pulse train, and electrical characteristics of the stimulation system (e.g., impedances of one or more circuits associated with respective electrodes). Even though parameter values may be selected, the device may or may not be able to output the pulse train according to the selected parameters. In other words, there may be situations in which the device cannot achieve a desired amplitude or pulse width due to one or more system constraints. Therefore, the system may indicate, via a graphical user interface, a maximum selectable value for an electrical stimulation parameter, such as an electrical current amplitude or a voltage amplitude, that may be available for individual electrodes (e.g., anodes/cathodes) and for the entire lead. As such, the system may indicate to the user that the device can, or cannot, output the pulse train according to the parameter values selected by the user. In some examples, the system may automatically adjust one or more parameters defining the different pulse trains based on evoked compound action potential (ECAP) signals sensed from a patient.

In one example, this disclosure describes a method including: obtaining, by processing circuitry, at least one first electrical stimulation parameter of an electrical stimulation program that defines an electrical stimulation therapy deliverable to a patient, determining, by processing circuitry, an electrical characteristic of a stimulation system configured to deliver the electrical stimulation therapy according to the electrical stimulation program; determining, by processing circuitry and based on the electrical characteristic and the at least one first electrical stimulation parameter, a maximum selectable value for a second electrical stimulation parameter of the electrical stimulation program; and outputting, for display by a user interface, the maximum selectable value.

In another example, this disclosure describes a system including: processing circuitry configured to: obtain a first electrical stimulation parameter of an electrical stimulation program that defines an electrical stimulation therapy deliverable to a patient, determine an electrical characteristic of a stimulation system configured to deliver the electrical stimulation therapy according to the electrical stimulation program; determine, based on the electrical characteristic and the at least one first electrical stimulation parameter, a maximum selectable value for a second electrical stimulation parameter of the electrical stimulation program; and output, for display by a user interface, the maximum selectable value.

In another example, this disclosure describes a non-transitory computer-readable medium including instructions that, when executed, control processing circuitry to: obtain a first electrical stimulation parameter of an electrical stimulation program that defines an electrical stimulation therapy deliverable to a patient, determine an electrical characteristic of a stimulation system configured to deliver the electrical stimulation therapy according to the electrical stimulation program; determine, based on the electrical characteristic and the at least one first electrical stimulation parameter, a maximum selectable value for a second electrical stimulation parameter of the electrical stimulation program; and output, for display by a user interface, the maximum selectable value.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages of the techniques will be apparent from the description and drawings, and from the claims.

### BRIEF DESCRIPTION OF DRAWINGS

FIG. 1 is a conceptual diagram illustrating an example system that includes a medical device programmer and an IMD, according to the techniques of the disclosure.

FIG. 2 is a block diagram of the example IMD of FIG. 1.

FIG. 3 is a block diagram of the example external programmer of FIG. 1.

FIG. 4 is a conceptual diagram illustrating an example user interface configured to display a maximum selectable value of one or more electrical stimulation parameters, according to the techniques of the disclosure.

FIG. 5 is a flowchart illustrating an example method for indicating a maximum selectable value of an electrical stimulation parameter within a user interface, in accordance with one or more techniques of this disclosure.

Like reference characters denote like elements throughout the description and figures.

### DETAILED DESCRIPTION

The disclosure describes examples of medical devices, systems, and techniques for adjusting electrical stimulation delivered to a patient based on a user input. Electrical stimulation therapy is typically delivered to a target tissue (e.g., nerves of the spinal cord or muscle) of a patient via two or more electrodes. One or more parameters of the electrical stimulation therapy (e.g., electrode combination, pulse width, pulse frequency, voltage or electrical current amplitude, and the like) are selected by a clinician and/or the patient in order to enable the system to generate electrical stimulation configured to provide relief from various symptoms, such as pain, nervous system disorders, muscle disorders, etc. However, over time, characteristics of the system and/or the patient may change, the previously satisfactory parameters of the electrical stimulation therapy may no longer be satisfactory. Accordingly, one or more parameter values may need to change in order for the stimulation to maintain efficacy for the patient. A patient, clinician, or other system user may interact with a patient programmer to program and/or reprogram the electrical stimulation parameters used by an electrical stimulation device, such as an implantable medical device (IMD). In some examples, the programmer may be configured to receive adjustments to a stimulation parameter, such as a change to the pulse width, frequency, or amplitude (electrical current or voltage), and/or a change to the number of electrodes (e.g., anodes and/or cathodes) that define the delivered electrical stimulation.

The ability of a system to output electrical stimulation according to selected parameter values, e.g., to output voltage or electrical current amplitude, may depend on electrical characteristics of the system. For example, the ability of the system to output electrical stimulation may depend on the electrical efficiency of the electrodes (configured as anodes or cathodes), the impedance of the electrodes, the impedance of the electrical stimulation system, charge density, the available electrical power, e.g., battery size/power, the battery life, e.g., the remaining charge of the battery, a recharge burden, and/or a device life (e.g., life of the components of the device).

For some systems, certain parameter values, such as higher amplitude values, may not be capable of being delivered from the system for certain electrodes or under certain parameter value combinations. Additionally, the ability of the system to output electrical stimulation according to a selected and/or desired parameter, e.g., a particular electrical current or voltage amplitude, may depend and/or change based on other parameters, e.g., a particular pulse frequency. For example, the maximum amplitude the system is able to output may depend on other parameters, e.g., pulse frequency, width, and/or electrode combination. In these cases, the only way a patient or clinician will know when the system cannot achieve one or more parameter values is by attempting to deliver stimulation according to those parameter values and the patient not receiving the expected stimulation. These events can make patient programming sessions frustrating and/or result an electrical stimulation program having electrical stimulation parameters near an edge and/or limit of the dynamic range available to the electrical stimulation device. At these limits, a patient may have minimal or no ability to increase (or decrease) these one or more parameters to a desired amount at a later time due to the limits of the current electrical stimulation parameters settings.

As described herein, systems, devices, and techniques may provide solutions to one or more of the above-referenced problems by indicating, e.g., via a graphical user interface, a maximum selectable value for an electrical stimulation parameter, such as an electrical current amplitude or a voltage amplitude, that may be available for individual anodes/cathodes and/or for an entire electrical stimulation lead. This maximum selectable value may be determined based on one or more other parameters selected for that electrical stimulation. As such, the system may indicate to the user available parameter values for the desired program before stimulation is even delivered. Therefore, the user can be notified of stimulation parameter values that can or cannot be achieved by the system. In some examples, the system may automatically adjust the values of the different pulse trains based on evoked compound action potential (ECAP) signals sensed from a patient. This maximum selectable value information, e.g., how much “head room” is available for adjusting one or more of the electrical stimulation therapy parameters, is important for the patient and/or clinician assisting the patient, and especially advantageous for “distance diagnostics,” e.g., over the phone conversations between clinician and patient, reprogramming sessions when the patient and clinician are in different locations, and the like.

In some examples, the systems, devices, and techniques may provide for suggesting parameter adjustments, and/or automatically adjusting one or more parameters, to accommodate a desired and/or user selected parameter, or the achieve the determined maximum selectable value of a parameter. If a patient and/or clinician selects a parameter value that is not achievable, but less than the maximum selectable value, the system may suggest changes to one or more other parameters, and/or automatically change one or more other parameters, to achieve the selected parameter value that is less than or equal to the maximum selectable value. For example, patient and/or clinician selects an electrode current amplitude value of 15 milliamperes (mA), for which the device may not be able to output with a currently selected frequency of 500 hertz (Hz), and pulse width of 300 microseconds, but is less than a maximum selectable value for the electrode current amplitude, the system may determine how to reach the currently selected value of 15 mA and output a notification with instructions and/or information on what changes to make to do so, or automatically make such changes. For example, the system may determine different frequencies and/or pulse widths in order to be able to output the 15 mA selected amplitude, and in some examples may determine frequencies and/or pulse widths in order to output the 15 mA while maintaining the “dose” (e.g., electrical stimulation and/or current delivered over a period of time, e.g., over at least multiple pulses) and/or maintaining a constant charge. For example, a system may not be able to output a patient/user clinician selected electrode current amplitude value of 10 mA at 600 Hz frequency with a 300 microsecond pulse width. The system may determine the maximum selectable electrode current amplitude is 10 mA or greater, and determine that the 10 mA may be output while maintaining a constant charge (e.g., charge per second) at a frequency of 200 Hz and 900 microsecond pulse width. In some examples, the system may automatically determine such parameters and output the information, or make the changes, or may output the information based on a patient/clinician query, e.g., determine one or more achievable parameters based on one or more other parameter values provided by the patient/clinician, and/or in some examples, one or more maximum selectable parameters.

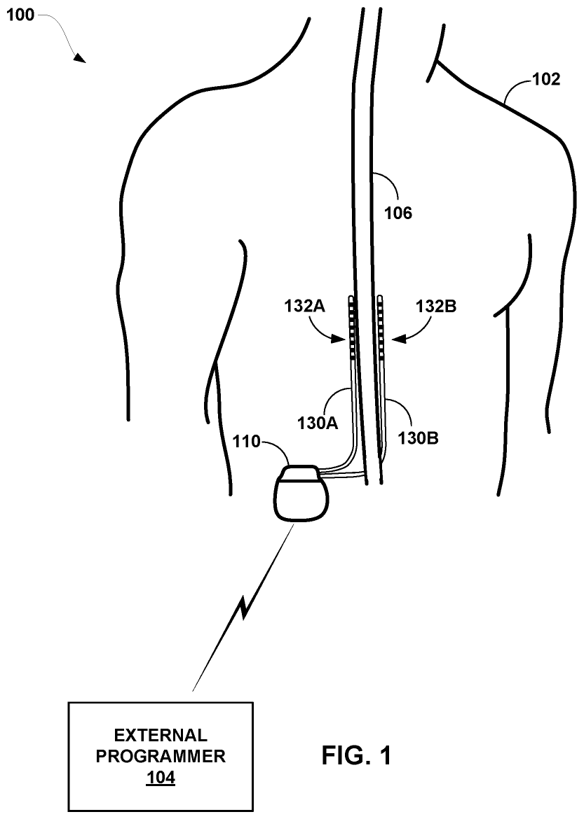

FIG. 1 is a conceptual diagram illustrating example system 100 that includes implantable medical device (IMD) 110 configured to deliver electrical stimulation therapy to patient 102. Although the techniques described in this disclosure are generally applicable to a variety of medical devices including external devices and IMDs, application of such techniques to IMDs and, more particularly, implantable electrical stimulators (e.g., neurostimulators) will be described for purposes of illustration. More particularly, the disclosure will refer to an implantable SCS system for purposes of illustration, but without limitation as to other types of medical devices or other therapeutic applications of medical devices.

As shown in FIG. 1, system 100 includes an IMD 110, leads 130A and 130B, and external programmer 104 shown in conjunction with a patient 102, who is ordinarily a human patient. In the example of FIG. 1, IMD 110 is an implantable electrical stimulator that is configured to generate and deliver electrical stimulation therapy to patient 102 via one or more electrodes 132A and/or 132B of leads 130A and/or 130B, respectively, (collectively, “electrodes 132 of leads 130”), e.g., for relief of chronic pain or other symptoms. In other examples, IMD 110 may be coupled to a single lead carrying multiple electrodes or more than two leads each carrying multiple electrodes. In some examples, the stimulation signals, or pulses (e.g., control pulses), may be configured to elicit detectable ECAP signals that IMD 110 may use to determine the posture state occupied by patient 102 and/or determine how to adjust one or more parameters that define stimulation therapy. IMD 110 may be a chronic electrical stimulator that remains implanted within patient 102 for weeks, months, or even years. In other examples, IMD 110 may be a temporary, or trial, stimulator used to screen or evaluate the efficacy of electrical stimulation for chronic therapy. In one example, IMD 110 is implanted within patient 102, while in another example, IMD 110 is an external device coupled to percutaneously implanted leads. In some examples, IMD 110 uses one or more leads, while in other examples, IMD 110 is leadless.

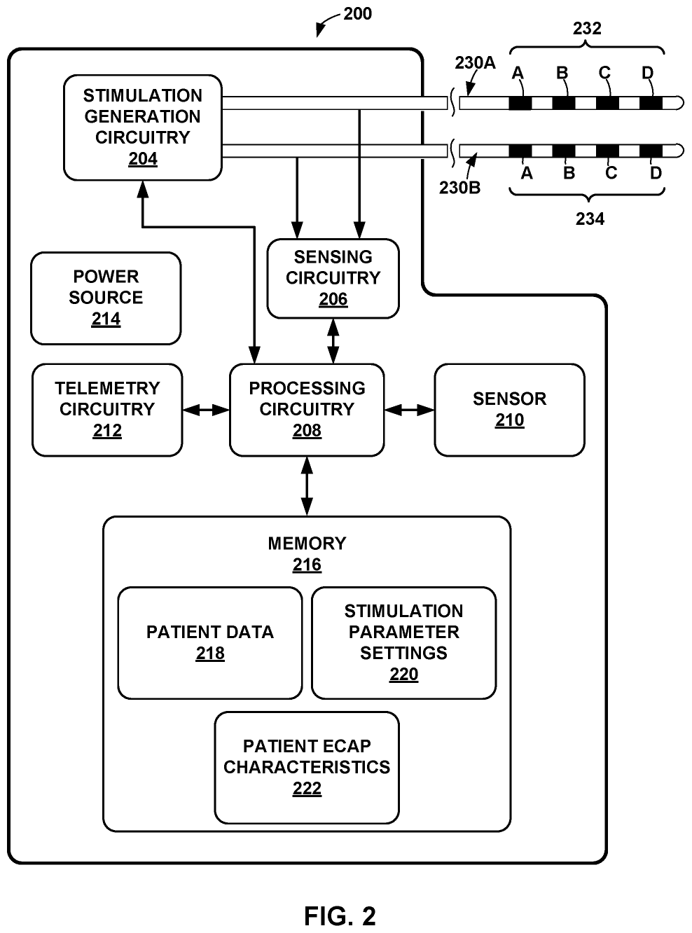

IMD 110 may be constructed of any polymer, metal, or composite material sufficient to house the components of IMD 110 (e.g., components illustrated in FIG. 2) within patient 102. In this example, IMD 110 may be constructed with a biocompatible housing, such as titanium or stainless steel, or a polymeric material such as silicone, polyurethane, or a liquid crystal polymer, and surgically implanted at a site in patient 102 near the pelvis, abdomen, or buttocks. In other examples, IMD 110 may be implanted within other suitable sites within patient 102, which may depend, for example, on the target site within patient 102 for the delivery of electrical stimulation therapy. The outer housing of IMD 110 may be configured to provide a hermetic seal for components, such as a rechargeable or non-rechargeable power source. In addition, in some examples, the outer housing of IMD 110 is selected from a material that facilitates receiving energy to charge the rechargeable power source.

Electrical stimulation energy, which may be constant electrical current or constant voltage-based pulses, for example, is delivered from IMD 110 to one or more target tissue sites of patient 102 via one or more electrodes 132 of implantable leads 130. In the example of FIG. 1, leads 130 carry electrodes 132 that are placed adjacent to the target tissue of spinal cord 106. One or more of the electrodes may be disposed at a distal tip of a lead 130 and/or at other positions at intermediate points along the lead. Leads 130 may be implanted and coupled to IMD 110. The electrodes 132 may transfer electrical stimulation generated by an electrical stimulation generator in IMD 110 to tissue of patient 102. Although leads 130 may each be a single lead, lead 130 may include a lead extension or other segments that may aid in implantation or positioning of lead 130. In some other examples, IMD 110 may be a leadless stimulator with one or more arrays of electrodes arranged on a housing of the stimulator rather than leads that extend from the housing. In addition, in some other examples, system 100 may include one lead or more than two leads, each coupled to IMD 110 and directed to similar or different target tissue sites.

The electrodes 132 of leads 130 may be electrode pads on a paddle lead, circular (e.g., ring) electrodes surrounding the body of the lead, conformable electrodes, cuff electrodes, segmented electrodes (e.g., electrodes disposed at different circumferential positions around the lead instead of a continuous ring electrode), any combination thereof (e.g., ring electrodes and segmented electrodes) or any other type of electrodes capable of forming unipolar, bipolar or multipolar electrode combinations for therapy. Ring electrodes arranged at different axial positions at the distal ends of lead 130 will be described for purposes of illustration.

The deployment of electrodes 132 via leads 130 is described for purposes of illustration, but arrays of electrodes may be deployed in different ways. For example, a housing associated with a leadless stimulator may carry arrays of electrodes, e.g., rows and/or columns (or other patterns), to which shifting operations may be applied. Such electrodes may be arranged as surface electrodes, ring electrodes, or protrusions. As a further alternative, electrode arrays may be formed by rows and/or columns of electrodes on one or more paddle leads. In some examples, electrode arrays include electrode segments, which are arranged at respective positions around a periphery of a lead, e.g., arranged in the form of one or more segmented rings around a circumference of a cylindrical lead. In other examples, one or more of leads 130 are linear leads having 8 ring electrodes 132 along the axial length of the lead. In another example, the electrodes are segmented rings arranged in a linear fashion along the axial length of the lead and at the periphery of the lead.

The stimulation parameter set of a stimulation program that defines the stimulation pulses of electrical stimulation therapy by IMD 110 through the electrodes 132 of leads 130 may include information identifying which electrodes 132 have been selected for delivery of stimulation according to a stimulation program, the polarities of the selected electrodes 132, i.e., the electrode combination for the program, voltage or electrical current amplitude, pulse frequency, pulse width, pulse shape of stimulation delivered by the electrodes 132. These stimulation parameters values that make up the stimulation parameter set that defines pulses may be predetermined parameter values defined by a user and/or automatically determined by system 100 based on one or more factors or user input.

Although FIG. 1 is directed to SCS therapy, e.g., used to treat pain, in other examples system 100 may be configured to treat any other condition that may benefit from electrical stimulation therapy. For example, system 100 may be used to treat tremor, Parkinson's disease, epilepsy, a pelvic floor disorder (e.g., urinary incontinence or other bladder dysfunction, fecal incontinence, pelvic pain, bowel dysfunction, or sexual dysfunction), obesity, gastroparesis, or psychiatric disorders (e.g., depression, mania, obsessive compulsive disorder, anxiety disorders, and the like). In this manner, system 100 may be configured to provide therapy taking the form of deep brain stimulation (DBS), peripheral nerve stimulation (PNS), peripheral nerve field stimulation (PNFS), cortical stimulation (CS), pelvic floor stimulation, gastrointestinal stimulation, or any other stimulation therapy capable of treating a condition of patient 102.

In some examples, leads 130 includes one or more sensors configured to allow IMD 110 to monitor one or more parameters of patient 102, such as patient activity, pressure, temperature, or other characteristics. The one or more sensors may be provided in addition to, or in place of, therapy delivery by leads 130.

IMD 110 is generally configured to deliver electrical stimulation therapy to patient 102 via selected combinations of electrodes 132 carried by one or both of leads 130, alone or in combination with an electrode carried by or defined by an outer housing of IMD 110. The target tissue for the electrical stimulation therapy may be any tissue affected by electrical stimulation, which may be in the form of electrical stimulation pulses or continuous waveforms. In some examples, the target tissue includes nerves, smooth muscle or skeletal muscle. In the example illustrated by FIG. 1, the target tissue is tissue proximate spinal cord 106, such as within an intrathecal space or epidural space of spinal cord 106, or, in some examples, adjacent nerves that branch off spinal cord 106. Leads 130 may be introduced into spinal cord 106 in via any suitable region, such as the thoracic, cervical or lumbar regions. Stimulation of spinal cord 106 may, for example, prevent pain signals from traveling through spinal cord 106 and to the brain of patient 102. Patient 102 may perceive the interruption of pain signals as a reduction in pain and, therefore, efficacious therapy results. In other examples, stimulation of spinal cord 106 may produce paresthesia which may be reduce the perception of pain by patient 102, and thus, provide efficacious therapy results. In some examples, stimulation of spinal cord 106 or other anatomical structures associated with the spinal cord (e.g., nerves and cells associated with the nervous system) may provide relief from symptoms that may not produce paresthesia. For example, IMD 110 may deliver stimulation with intensities (e.g., values of amplitude and/or pulse width) below a sensory or perception threshold (e.g., sub-threshold stimulation) that reduces pain without paresthesia. In multimodal stimulation (e.g., differential targeted multiplexed stimulation), for example, IMD 110 may deliver one pulse train at a higher frequency via one electrode combination and a second pulse train on an interleaved basis with a lower frequency via a second electrode combination, where both pulse trains are delivered at a sub-threshold intensity.

IMD 110 is configured to generate and deliver electrical stimulation therapy to a target stimulation site within patient 102 via the electrodes 132 of leads 130 to patient 102 according to one or more therapy stimulation programs. A therapy stimulation program defines values for one or more parameters (e.g., a parameter set) that define an aspect of the therapy delivered by IMD 110 according to that program. For example, a therapy stimulation program that controls delivery of stimulation by IMD 110 in the form of pulses may define values for voltage or electrical current pulse amplitude, pulse width, pulse rate (e.g., pulse frequency), electrode combination, pulse shape, etc. for stimulation pulses delivered by IMD 110 according to that program. In some examples, one or more therapy stimulation programs define multiple different pulse trains that have different parameter values (e.g., different pulse frequencies, amplitudes, pulse widths, and/or electrode combinations) but are delivered on an interleaved basis to together provide a therapy for the patient.

A user, such as a clinician or patient 102, may interact with a user interface of an external programmer 104 to program IMD 110. Programming of IMD 110 may refer generally to the generation and transfer of commands, programs, or other information to control the operation of IMD 110. In this manner, IMD 110 may receive the transferred commands and programs from external programmer 104 to control stimulation, such as stimulation pulses that provide electrical stimulation therapy. For example, external programmer 104 may transmit therapy stimulation programs, stimulation parameter adjustments, therapy stimulation program selections, posture states, user input, or other information to control the operation of IMD 110, e.g., by wireless telemetry or wired connection.

In some cases, external programmer 104 may be characterized as a physician or clinician programmer if it is primarily intended for use by a physician or clinician. In other cases, external programmer 104 may be characterized as a patient programmer if it is primarily intended for use by a patient. A patient programmer may be generally accessible to patient 102 and, in many cases, may be a portable device that may accompany patient 102 throughout the patient's daily routine. For example, a patient programmer may receive input from patient 102 when the patient wishes to terminate or change electrical stimulation therapy, or when a patient perceives stimulation being delivered. For example, external programmer 104 may receive user input to change a stimulation parameter, and external programmer 104 or IMD 110 may adjust the values of the parameter for multiple different pulse trains (e.g., pulses delivered by different electrode combinations) to maintain a ratio of the parameter values between the multiple different pulse trains. In general, a physician or clinician programmer may support selection and generation of programs by a clinician for use by IMD 110, whereas a patient programmer may support adjustment and selection of such programs by a patient during ordinary use. In other examples, external programmer 104 may include, or be part of, an external charging device that recharges a power source of IMD 110. In this manner, a user may program and charge IMD 110 using one device, or multiple devices.

As described herein, information may be transmitted between external programmer 104 and IMD 110. Therefore, IMD 110 and external programmer 104 may communicate via wireless communication using any techniques known in the art. Examples of communication techniques may include, for example, radiofrequency (RF) telemetry and inductive coupling, but other techniques are also contemplated. In some examples, external programmer 104 includes a communication head that may be placed proximate to the patient's body near the IMD 110 implant site to improve the quality or security of communication between IMD 110 and external programmer 104. Communication between external programmer 104 and IMD 110 may occur during power transmission or separate from power transmission.

In some examples, IMD 110, in response to commands from external programmer 104, delivers electrical stimulation therapy according to a plurality of therapy stimulation programs to a target tissue site of the spinal cord 106 of patient 102 via electrodes 132 on leads 130. In some examples, IMD 110 modifies therapy stimulation programs as therapy needs of patient 102 evolve over time. For example, the modification of the therapy stimulation programs may cause the adjustment of at least one parameter of the plurality of stimulation pulses. When patient 102 receives the same therapy for an extended period, the efficacy of the therapy may be reduced. In some cases, parameters of the plurality of stimulation pulses may be automatically updated.

Efficacy of electrical stimulation therapy may be indicated by patient feedback, e.g., received via external programmer 104, or alternatively or additionally by one or more characteristics of an action potential that is evoked by a pulse delivered by IMD 110, i.e., a characteristic value of an evoked compound action potential (ECAP) signal. In some examples, efficacy of electrical stimulation therapy may be alternatively or additionally indicated by other local field potentials and/or biological signals that may directly or indirectly correlate to a patient's pain state or overall well-being. For example, local field potentials may indicate actual pain signals, heart rate, respiration, or the like, and may correlate to overall wellbeing, and also may correlate to the effectiveness and/or efficacy of the electrical stimulation therapy. Electrical stimulation therapy delivery by leads 130 of IMD 110 may cause neurons within the target tissue to evoke a compound action potential that travels up and down the target tissue, eventually arriving at sensing electrodes of IMD 110. Furthermore, stimulation may also elicit at least one ECAP signal, and ECAPs responsive to stimulation may also be a surrogate for the effectiveness of the therapy. The amount of action potentials (e.g., number of neurons propagating action potential signals) that are evoked may be based on the various parameters of electrical stimulation pulses such as amplitude, pulse width, frequency, pulse shape (e.g., slew rate at the beginning and/or end of the pulse), etc. The slew rate may define the rate of change of the voltage and/or electrical current amplitude of the control pulse at the beginning and/or end of each control pulse or each phase within the pulse. For example, a very high slew rate indicates a steep or even near vertical edge of the pulse, and a low slew rate indicates a longer ramp up (or ramp down) in the amplitude of the pulse. In some examples, these parameters contribute to an intensity of the electrical stimulation. In addition, a characteristic of the ECAP signal (e.g., an amplitude) may change based on the distance between the stimulation electrodes and the nerves subject to the electrical field produced by the delivered pulses.

In the example of FIG. 1, IMD 110 is described as performing a plurality of processing and computing functions. However, external programmer 104 instead may perform one, several, or all of these functions. In this alternative example, IMD 110 functions to relay sensed signals to external programmer 104 for analysis, and external programmer 104 transmits instructions to IMD 110 to adjust the one or more parameters defining the electrical stimulation therapy based on analysis of the sensed signals. For example, IMD 110 may relay the sensed signal indicative of an ECAP to external programmer 104. External programmer 104 may compare the parameter value of the ECAP to the target ECAP characteristic value, and in response to the comparison, external programmer 104 may instruct IMD 110 to adjust one or more stimulation parameter that defines the electrical stimulation pulses delivered to patient 102.

In some examples, the system changes the target ECAP characteristic value and/or growth rate(s) over a period of time, such as according to a change to a stimulation threshold (e.g., a perception threshold or detection threshold specific for the patient). The system may be programmed to change the target ECAP characteristic in order to adjust the intensity of informed pulses to provide varying sensations to the patient (e.g., increase or decrease the volume of neural activation). Although the system may change the target ECAP characteristic value, received ECAP signals may still be used by the system to adjust one or more parameter values of the pulses in order to meet the target ECAP characteristic value.

One or more devices within system 100, such as IMD 110 and/or external programmer 104, may perform various functions as described herein. For example, IMD 110 may include stimulation circuitry configured to deliver electrical stimulation, and sensing circuitry configured to sense a plurality ECAP signals, and processing circuitry. The processing circuitry may be configured to control the stimulation circuitry to deliver a plurality of electrical stimulation pulses having different amplitude values and control the sensing circuitry to detect, after delivery of each electrical stimulation pulse of the plurality of electrical stimulation pulses, a respective ECAP signal of the plurality of ECAP signals.

As described herein, IMD 110 may modulate or adjust one or more stimulation parameters that at least partially define electrical stimulation based on a sensed ECAP signals to employ a closed-loop feedback system for adjusting stimulation parameters that define electrical stimulation pulses. In one example, IMD 110 includes stimulation generation circuitry configured to generate and deliver electrical stimulation to patient 102 according one or more sets of stimulation parameters that at least partially define the respective pulses of the electrical stimulation (a set of electrical stimulation parameters may include one or more electrical stimulation parameters). Each set of stimulation parameters may include at least one of an electrode combination, an electrical current amplitude or a voltage amplitude, a pulse width, a pulse frequency, or a pulse shape.

As described herein, IMD 110 may be configured to obtain a first electrical stimulation parameter of an electrical stimulation program that defines an electrical stimulation therapy deliverable to a patient. For example, a clinician or patient 102 may interact with a user interface of external programmer 104 to select an electrical stimulation program defining the electrical stimulation parameters, including the first electrical stimulation parameter. IMD 110 may receive the electrical stimulation program from external programmer 104.

IMD 110 may be configured to determine an electrical characteristic of a stimulation system configured to deliver the electrical stimulation therapy according to the electrical stimulation program. For example, IMD 110 may be configured to determine an electrical characteristic such as an impedance, an electrical efficiency, a charge density, a battery life, a device life, a recharge burden, or the like, associated with IMD 110 and other components of the system in some examples. In some examples, IMD 110 may be configured to determine an impedance of each electrode 132 (e.g., anodes and/or cathodes) of leads 130, or the impedance of one or more circuits associated with each electrode 132, included in the electrical stimulation program, e.g., impedances of the electrodes of the electrode combination defined by the electrical stimulation program. IMD 110 may be configured to determine an electrical efficiency of each electrode 132 included in the electrical stimulation program. The electrical efficiency may be representative of the output power required to be delivered via each electrode 130 to achieve efficacious therapy in relation to the electrical power losses (e.g., due to impedance) or delivery of electrical stimulation of tissue of patient 102 that does not result in efficacious treatment and/or results.

In some examples, IMD 110 may be configured to determine an electrical characteristic such as a charge density of one or more of electrodes 132. For example, IMD 110 may determine a charge density based on an electrode surface area, electrode impedance, and/or a current through (or a voltage across) patient tissue between electrodes 132, e.g., a number of electrons through tissue per time. In some examples, IMD 110 may be configured to determine an electrical characteristic based on one or more standard model of electrode characteristics, e.g., an allowed charge density of electrodes 132 based on an accepted industry or safety standard.

In some examples, IMD 110 may be configured to determine an electrical characteristic such as a battery capacity, size, and/or power, e.g., a number of ampere-hours (or milliampere-hours) or watt-hours (or milliwatt-hours). In some examples, IMD 110 may be configured to determine an electrical characteristic such as a battery life, e.g., how long the battery may provide power (e.g., the way the battery is discharged may impact the actual battery life versus the capacity in milliamp hours). In some examples, IMD 110 may be configured to determine an electrical characteristic such as a recharge burden, e.g., an amount of time to charge the battery and/or the number of uses of IMD 110 before the battery needs to be recharged.

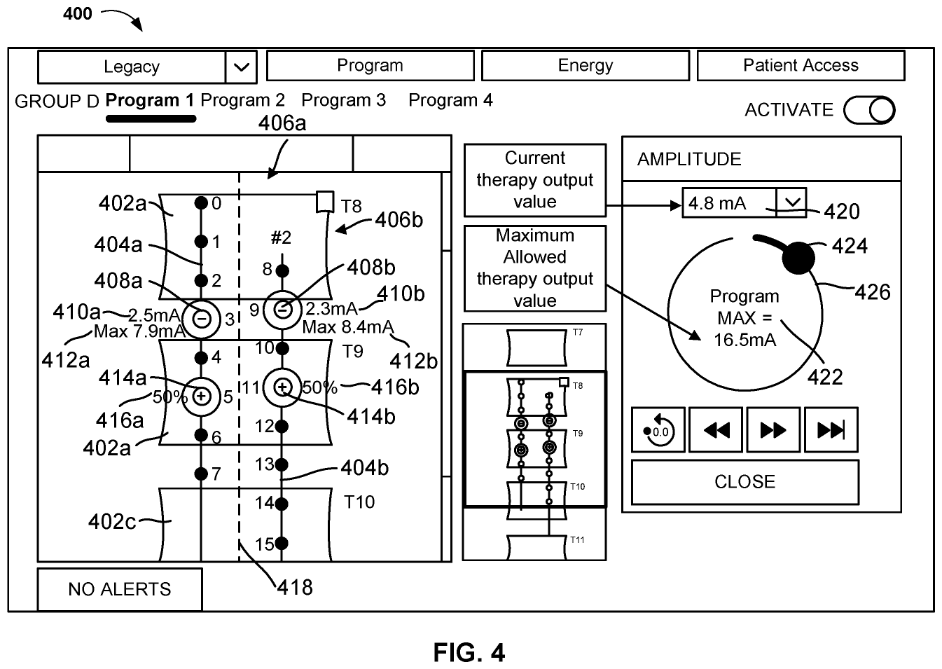

In some examples, IMD 110 may be configured to determine an electrical characteristic such as a lead location, upon which a stimulation circuit may depend. For example, the axial (along midline 418 shown in FIG. 4) and lateral (perpendicular to midline 418 shown in FIG. 4) locations of one or more leads, relative to an electrode of another lead and/or tissue of the patient, may change the overall impedance of the electrical stimulation therapy system. For example, IMD 110 may be able to deliver a first amount of electrical stimulation (e.g., a first intensity) at a first location, but a different amount of electrical stimulation (e.g., a second and different intensity) if the lead is moved, relative to patient anatomy and/or another lead or electrode 132.

In some examples, IMD 110 may be configured to determine an electrical characteristic such as a device life. For example, the operational lifetime of IMD 110 may correspond to the function of one or more electrical components of the device (circuit board, battery, wires and/or connectors, individual resistors, capacitors, inductors, integrated circuit chips, leads 130 or electrodes 132, or the like). IMD 110 may determine an electrical characteristics based at least in part on a determined and/or projected device life of IMD 110.

IMD 110 may be configured to determine a maximum selectable value for a second electrical stimulation parameter of the electrical stimulation program based on at least one electrical characteristic and the at least one first electrical stimulation parameter. For example, IMD 110 may be configured to determine a maximum selectable voltage amplitude (e.g., a second electrical stimulation parameter) of a particular electrode 132, or of all of electrodes 132, of the electrode combination defined by the electrical stimulation program based on the impedance of the electrodes 132 for that electrode combination and at least one other parameter value, such as the pulse frequency (e.g., the first electrical stimulation parameter). In some examples, IMD 110 may be configured to determine a maximum selectable value for one or more electrical stimulation parameters based on one or more other electrical stimulation parameters. For example, IMD 110 may be configured to determine a maximum selectable voltage amplitude for each electrode 132 based on the impedance and/or efficiency of each electrode 132 and any other electrical stimulation parameter, alone or in combination, e.g., various pulse widths, pulse frequencies, and electrode combinations. As another example, IMD 110 maybe be configured to determine a maximum selectable pulse frequency based on the impedance and/or efficiency of each electrode 132 and any other electrical stimulation parameter, alone or in combination, e.g., various pulse widths, amplitudes, and electrode combinations.

IMD 110 may be configured to output the maximum selectable values of the one or more electrical stimulation parameters for display by a user interface. For example, IMD 110 may output the maximum selectable value(s) to external programmer 104 for display in a user interface of external programmer 104. In other words, IMD 110 and/or external programmer 104 may be configured to convey to the user, e.g., clinician and/or patient, the current therapy setting(s) in relation to the maximum therapy setting(s) available for each of the electrical stimulation parameters, e.g., the maximum selectable frequency, pulse width, voltage or electrical current amplitude, or number of anodes and/or cathodes. The maximum selectable value may thus be dependent on one or more factors related to the specific stimulation program. The maximum selectable value may be lower than published system limits or lower for certain stimulation parameter sets when compared to other stimulation parameter sets. For example, the system may be capable of delivering 20 mA of current to an electrode combination, but, due to one or more parameter values such as electrode combination or pulse frequency, the system may determine that the maximum selectable value for the selected stimulation parameter set is lower than 20 mA (e.g., 10 mA, 13 mA, 17 mA, etc.).

Although in one example IMD 110 takes the form of an SCS device, in other examples, IMD 110 takes the form of any combination of deep brain stimulation (DBS) devices, implantable cardioverter defibrillators (ICDs), pacemakers, cardiac resynchronization therapy devices (CRT-Ds), left ventricular assist devices (LVADs), implantable sensors, orthopedic devices, or drug pumps, as examples.

FIG. 2 is a block diagram of IMD 200. IMD 200 may be an example of IMD 110 of FIG. 1. In the example shown in FIG. 2, IMD 200 includes stimulation generation circuitry 204, sensing circuitry 206, processing circuitry 208, sensor 210, telemetry circuitry 212, power source 214, and memory 216. Each of these circuits may be or include programmable or fixed function circuitry can perform the functions attributed to respective circuitry. For example, processing circuitry 208 may include fixed-function or programmable circuitry, stimulation generation circuitry 204 may include circuitry can generate electrical stimulation signals such as pulses or continuous waveforms on one or more channels, sensing circuitry 206 may include sensing circuitry for sensing signals, and telemetry circuitry 212 may include telemetry circuitry for transmission and reception of signals. Memory 216 may store computer-readable instructions that, when executed by processing circuitry 208, cause IMD 200 to perform various functions described herein. Memory 216 may be a storage device or other non-transitory medium.

In the example shown in FIG. 2, memory 216 stores patient data 218, which may include anything related to the patient such as one or more patient postures, an activity level, or a combination of patient posture and activity level. Memory 216 may store stimulation parameter settings 220 within memory 216 or separate areas within memory 216. Each stored stimulation parameter setting 220 defines values for one or more electrical stimulation parameters and/or one or more sets of electrical stimulation parameters, e.g., pulse amplitude, pulse width, pulse frequency, electrode combination, pulse burst rate, pulse burst duration, and/or waveform shape (a set of electrical stimulation parameters may include one or more electrical stimulation parameters). Stimulation parameter settings 220 may also include additional information such as instructions regarding delivery of electrical stimulation signals based on stimulation parameter relationship data, which can include relationships between two or more stimulation parameters based upon data from electrical stimulation signals delivered to patient 102 or data transmitted from external programmer 104. The stimulation parameter relationship data may include measurable aspects associated with stimulation, such as an ECAP characteristic value. Stimulation parameter settings 220, or another portion of memory 216, may include instructions on how processing circuitry 208 can modulate informed stimulation parameters and/or control stimulation parameters based on the detected posture state and/or at least one of a target ECAP characteristic value or a threshold ECAP characteristic value, as described herein. Memory 216 may also include other values for one or more programs, such as the maximum selectable value for one or more parameters of the program.

Memory 216 also stores patient ECAP characteristics 222 which may include target ECAP characteristics and/or threshold ECAP characteristic values determined for the patient and/or a history of measured ECAP characteristic values for the patient. Memory 216 may include gain values that processing circuitry 208 may use to modulate informed and/or control stimulation pulses as described herein.

Accordingly, in some examples, stimulation generation circuitry 204 generates electrical stimulation signals (e.g., informed pulses and control pulses) in accordance with the electrical stimulation parameters noted above. Other ranges of stimulation parameter values may also be useful and may depend on the target stimulation site within patient 102. While stimulation pulses are described, stimulation signals may be of any form, such as continuous-time signals (e.g., sine waves) or the like. In some examples, IMD 200 may include switch circuitry (not shown) that may include one or more switch arrays, one or more multiplexers, one or more switches (e.g., a switch matrix or other collection of switches), or other electrical circuitry configured to direct stimulation signals from stimulation generation circuitry 204 to one or more of electrodes 232, 234, or directed sensed signals from one or more of electrodes 232, 234 to sensing circuitry 206. In other examples, stimulation generation circuitry 204 and/or sensing circuitry 206 may include sensing circuitry to direct signals to and/or from one or more of electrodes 232, 234, which may or may not also include switch circuitry.

Sensing circuitry 206 may be configured to monitor signals from any combination of electrodes 232, 234. In some examples, sensing circuitry 206 includes one or more amplifiers, filters, and analog-to-digital converters. Sensing circuitry 206 may be used to sense physiological signals, such as ECAPs. In some examples, sensing circuitry 206 detects ECAPs from a particular combination of electrodes 232, 234. In some cases, the particular combination of electrodes for sensing ECAPs includes different electrodes than a set of electrodes 232, 234 used to deliver control stimulation pulses and/or informed stimulation pulses. Alternatively, in other cases, the particular combination of electrodes used for sensing ECAPs includes at least one of the same electrodes as a set of electrodes used to deliver informed and/or control stimulation pulses to patient 102. Sensing circuitry 206 may provide signals to an analog-to-digital converter, for conversion into a digital signal for processing, analysis, storage, or output by processing circuitry 208.

Processing circuitry 208 may include any one or more of a microprocessor, a controller, a digital signal processor (DSP), an application specific integrated circuit (ASIC), a field-programmable gate array (FPGA), discrete logic circuitry, or any other processing circuitry can provide the functions attributed to processing circuitry 208 herein may be embodied as firmware, hardware, software or any combination thereof. Processing circuitry 208 controls stimulation generation circuitry 204 to generate electrical stimulation signals according to stimulation parameter settings 220 stored in memory 216 to apply stimulation parameter values, such as pulse amplitude, pulse width, pulse frequency, and waveform shape of each of the electrical stimulation signals.

In the example shown in FIG. 2, the set of electrodes 232 includes electrodes 232A, 232B, 232C, and 232D, and the set of electrodes 234 includes electrodes 234A, 234B, 234C, and 234D. In other examples, a single lead may include all eight electrodes 232 and 234 along a single axial length of the lead. Processing circuitry 208 also controls stimulation generation circuitry 204 to generate and apply the electrical stimulation signals to selected combinations of electrodes 232, 234. In some examples, stimulation generation circuitry 204 includes a switch circuit that may couple stimulation signals to selected conductors within leads 230, which, in turn, deliver the stimulation signals across selected electrodes 232, 234. Such a switch circuit may be a switch array, switch matrix, multiplexer, or any other type of switch circuitry can selectively couple stimulation energy to selected electrodes 232, 234 and to selectively sense bioelectrical neural signals of a spinal cord of the patient (not shown in FIG. 2) with selected electrodes 232, 234.

In other examples, however, stimulation generation circuitry 204 does not include a switch circuit and switch circuitry does not interface between stimulation generation circuitry 204 and electrodes 232, 234. In these examples, stimulation generation circuitry 204 comprises a plurality of individual and/or pairs of voltage sources, current sources, voltage sinks, or current sinks connected to each of electrodes 232, 234 such that each individual electrode and/or pairs of electrodes has a unique signal circuit. In other words, in these examples, each of electrodes 232, 234 is independently controlled via its own signal circuit (e.g., via a combination of a regulated voltage source and sink or regulated current source and sink), as opposed to switching signals between electrodes 232, 234.

Electrodes 232, 234 on respective leads 230 may be constructed of a variety of different designs. For example, one or both of leads 230 may include one or more electrodes at each longitudinal location along the length of the lead, such as one electrode at different perimeter locations around the perimeter of the lead at each of the locations A, B, C, and D. In one example, the electrodes may be electrically coupled to stimulation generation circuitry 204, e.g., via switch circuitry 202 and/or switch circuitry of the stimulation generation circuitry 204, via respective wires that are straight or coiled within the housing of the lead and run to a connector at the proximal end of the lead. In another example, each of the electrodes of the lead may be electrodes deposited on a thin film. The thin film may include an electrically conductive trace for each electrode that runs the length of the thin film to a proximal end connector. The thin film may then be wrapped (e.g., a helical wrap) around an internal member to form the lead 230. These and other constructions may be used to create a lead with a complex electrode geometry.

Although sensing circuitry 206 is incorporated into a common housing with stimulation generation circuitry 204 and processing circuitry 208 in FIG. 2, in other examples, sensing circuitry 206 may be in a separate housing from IMD 200 and may communicate with processing circuitry 208 via wired or wireless communication techniques.

In some examples, one or more of electrodes 232 and 234 may be suitable for sensing ECAPs. For instance, electrodes 232 and 234 may sense the voltage amplitude of a portion of the ECAP signals, where the sensed voltage amplitude is a characteristic the ECAP signal.

Memory 216 may be configured to store information within IMD 200 during operation. Memory 216 may include a computer-readable storage medium or computer-readable storage device. In some examples, memory 216 includes one or more of a short-term memory or a long-term memory. Memory 216 may include, for example, random access memories (RAM), dynamic random access memories (DRAM), static random access memories (SRAM), magnetic discs, optical discs, flash memories, or forms of electrically programmable memories (EPROM) or electrically erasable and programmable memories (EEPROM). In some examples, memory 216 is used to store data indicative of instructions for execution by processing circuitry 208. As discussed herein, memory 216 can store patient posture state data 218, stimulation parameter settings 220, and patient ECAP characteristics 222.

Sensor 210 may include one or more sensing elements that sense values of a respective patient parameter. As described, electrodes 232 and 234 may be the electrodes that sense, via sensing circuitry 206, a value of the ECAP indicative of a target stimulation intensity at least partially caused by a set of control stimulation parameter values. Sensor 210 may include one or more accelerometers, optical sensors, chemical sensors, temperature sensors, pressure sensors, or any other types of sensors. Sensor 210 may output patient parameter values that may be used as feedback to control delivery of electrical stimulation signals. IMD 200 may include additional sensors within the housing of IMD 200 and/or coupled via one of leads 108 or other leads. In addition, IMD 200 may receive sensor signals wirelessly from remote sensors via telemetry circuitry 212, for example. In some examples, one or more of these remote sensors may be external to patient (e.g., carried on the external surface of the skin, attached to clothing, or otherwise positioned external to the patient). In some examples, signals from sensor 210 may indicate a posture state (e.g., sleeping, awake, sitting, standing, or the like), and processing circuitry 208 may select target and/or threshold ECAP characteristic values according to the indicated posture state.

Telemetry circuitry 212 supports wireless communication between IMD 200 and an external programmer (not shown in FIG. 2) or another computing device under the control of processing circuitry 208. Processing circuitry 208 of IMD 200 may receive, as updates to programs, values for various stimulation parameters such as amplitude and electrode combination (e.g., for informed and/or control pulses), from the external programmer via telemetry circuitry 212. Updates to stimulation parameter settings 220 and input efficacy threshold settings 226 may be stored within memory 216. Telemetry circuitry 212 in IMD 200, as well as telemetry circuits in other devices and systems described herein, such as the external programmer, may accomplish communication by radiofrequency (RF) communication techniques. In addition, telemetry circuitry 212 may communicate with an external medical device programmer (not shown in FIG. 2) via proximal inductive interaction of IMD 200 with the external programmer. The external programmer may be one example of external programmer 104 of FIG. 1. Accordingly, telemetry circuitry 212 may send information to the external programmer on a continuous basis, at periodic intervals, or upon request from IMD 110 or the external programmer.

Power source 214 delivers operating power to various components of IMD 200. Power source 214 may include a rechargeable or non-rechargeable battery and a power generation circuit to produce the operating power. Recharging may be accomplished through proximal inductive interaction between an external charger and an inductive charging coil within IMD 200. In other examples, traditional primary cell batteries may be used. In some examples, processing circuitry 208 may monitor the remaining charge (e.g., voltage) of power source 214 and select stimulation parameter values that may deliver similarly effective therapy at lower power consumption levels when needed to extend the operating time of power source 214. For example, power source 214 may switch to a lower pulse frequency based on the relationships of parameters that may provide similar ECAP characteristic values.

According to the techniques of the disclosure, stimulation generation circuitry 204 of IMD 200 receives, via telemetry circuitry 212, instructions to deliver electrical stimulation according to stimulation parameter settings 220 to a target tissue site of the spinal cord of the patient via a plurality of electrode combinations of electrodes 232, 234 of leads 230 and/or a housing of IMD 200. Each electrical stimulation signal may elicit an ECAP that is sensed by sensing circuitry 206 via electrodes 232 and 234. Processing circuitry 208 may receive, via an electrical signal sensed by sensing circuitry 206, information indicative of an ECAP signal (e.g., a numerical value indicating a characteristic of the ECAP in electrical units such as voltage or power) produced in response to the electrical stimulation signal(s). Stimulation parameter settings 220 may be updated according to the ECAPs recorded at sensing circuitry 206 according to the following techniques.

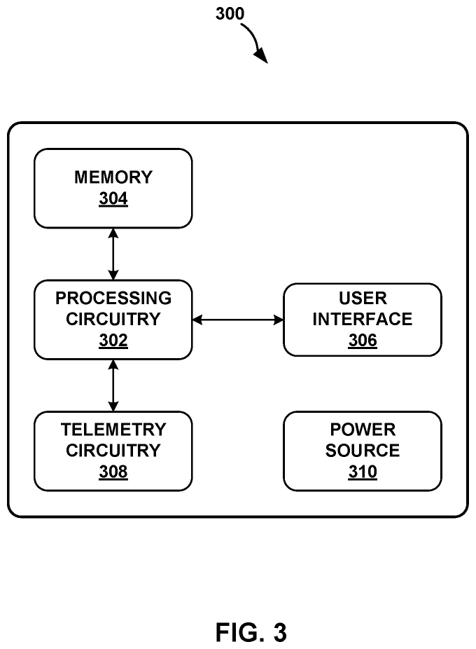

FIG. 3 is a block diagram of the example external programmer 300. External programmer 300 may be an example of external programmer 104 of FIG. 1. Although programmer 300 may generally be described as a hand-held device, external programmer 300 may be a larger portable device or a more stationary device. In addition, in some examples, external programmer 300 may be included as part of an external charging device or include the functionality of an external charging device. As illustrated in FIG. 3, external programmer 300 may include a processing circuitry 302, memory 304, user interface 306, telemetry circuitry 308, and power source 310. Storage device 304 may store instructions that, when executed by processing circuitry 302, cause processing circuitry 302 and external programmer 300 to provide the functionality ascribed to external programmer 300 throughout this disclosure. Each of these components, circuitry, or modules, may include electrical circuitry that can perform some, or all of the functionality described herein. For example, processing circuitry 302 may include processing circuitry to perform the processes discussed with respect to processing circuitry 302.

In general, programmer 300 comprises any suitable arrangement of hardware, alone or in combination with software and/or firmware, to perform the techniques attributed to programmer 300, and processing circuitry 302, user interface 306, and telemetry circuitry 308 of programmer 300. In various examples, programmer 300 may include one or more processors, such as one or more microprocessors, DSPs, ASICs, FPGAs, or any other equivalent integrated or discrete logic circuitry, as well as any combinations of such components. Programmer 300 also, in various examples, may include a memory 304, such as RAM, ROM, PROM, EPROM, EEPROM, flash memory, a hard disk, a CD-ROM, comprising executable instructions for causing the one or more processors to perform the actions attributed to them. Moreover, although processing circuitry 302 and telemetry circuitry 308 are described as separate, in some examples, processing circuitry 302 and telemetry circuitry 308 are functionally integrated. In some examples, processing circuitry 302 and telemetry circuitry 308 correspond to individual hardware units, such as ASICs, DSPs, FPGAs, or other hardware units.

Memory 304 (e.g., a storage device) may store instructions that, when executed by processing circuitry 302, cause processing circuitry 302 and programmer 300 to provide the functionality ascribed to programmer 300 throughout this disclosure. For example, memory 304 may include instructions that cause processing circuitry 302 to obtain a stimulation parameter setting from memory or receive a user input and send a corresponding command to programmer 300, or instructions for any other functionality. In addition, memory 304 may include a plurality of stimulation parameter settings, where each setting includes a parameter set that defines electrical stimulation. Memory 304 may also include instructions that control processing circuitry 304 to determine a maximum selectable value for one or more parameters as described herein. In some examples, memory 304 may include stimulation parameter setting 220 described above. Memory 304 may also store data received from a medical device (e.g., IMD 110). For example, memory 304 may store ECAP related data recorded at a sensing circuitry of the medical device, and in some examples memory 304 may include patient ECAP characteristics 222 described above. Memory 304 may also store data from one or more sensors of the medical device. In some examples, memory 304 may also store patient data 218 described above.

User interface 306 may include a button or keypad, lights, a speaker for voice commands, a display, such as a liquid crystal (LCD), light-emitting diode (LED), or organic light-emitting diode (OLED). In some examples the display may be a touch screen. User interface 306 can display any information related to the delivery of electrical stimulation, identified patient behaviors, sensed patient parameter values, patient behavior criteria, or any other such information. External programmer 300 may receive user input (e.g., indication of when the patient changes posture states) via user interface 306. The input may be, for example, in the form of pressing a button on a keypad or selecting an icon from a touch screen. The input may request starting or stopping electrical stimulation, the input may request a new spatial electrode movement pattern or a change to an existing spatial electrode movement pattern, of the input may request some other change to the delivery of electrical stimulation. In some examples, user interface 306 may receive user input requesting to adjust a stimulation parameter, and external programmer 300 or IMD 110, for example, may adjust the value of the stimulation parameter for multiple pulse trains in order to maintain a ratio of the parameter after the adjustment for the multiple pulse trains defined by different parameter values. In other examples, user interface 306 may receive input from the patient and/or clinician regarding efficacy of the therapy, such as binary feedback, numerical ratings, textual input, etc. In some examples, processing circuitry 302 may interpret patient requests to change therapy as negative feedback regarding the current parameter values used to define therapy. User interface 306 may be configured to have the functionality ascribed to user interface 400 of FIG. 4, described below. For example, user interface 306 may display a representation of the maximum selectable value for a stimulation parameter and/or receive user input adjusting the value of the parameter within a range defined at least partially by the maximum selectable value.

Telemetry circuitry 308 may support wireless communication between the medical device and programmer 300 under the control of processing circuitry 302. Telemetry circuitry 308 can communicate with another computing device via wireless communication techniques, or direct communication through a wired connection. In some examples, telemetry circuitry 308 provides wireless communication via an RF or proximal inductive medium. In some examples, telemetry circuitry 308 includes an antenna, which may take on a variety of forms, such as an internal or external antenna.

Examples of local wireless communication techniques that may be employed to facilitate communication between programmer 300 and IMD 110 include RF communication according to the 902.11 or Bluetooth specification sets or other standard or proprietary telemetry protocols. In this manner, other external devices may be capable of communicating with programmer 300 without needing to establish a secure wireless connection. As described herein, telemetry circuitry 308 can transmit a spatial electrode movement pattern or other stimulation parameter values to IMD 110 for delivery of electrical stimulation.

In some examples, selection of stimulation parameter settings may be transmitted to the medical device for delivery to the patient. In other examples, stimulation parameter settings may include medication, activities, or other instructions that the patient must perform themselves or a caregiver perform for patient 102. In some examples, external programmer 300 may provide visual, audible, and/or tactile notifications that indicate there are new instructions. External programmer 300 may require receiving user input acknowledging that the instructions have been completed in some examples.

Power source 310 can deliver operating power to various components of programmer 300. Power source 310 may be the same as or substantially similar to power source 214. Power source 310 may include a battery and a power generation circuit to produce the operating power. In some examples, the battery is rechargeable to allow extended operation. Recharging may be accomplished by electrically coupling power source 310 to a cradle or plug that is connected to an alternating current (AC) outlet. In addition, recharging may be accomplished through proximal inductive interaction between an external charger and an inductive charging coil within external programmer 300. In other examples, traditional batteries (e.g., nickel cadmium or lithium ion batteries) may be used. In addition, external programmer 300 may be directly coupled to an alternating current outlet to operate.

The architecture of external programmer 300 illustrated in FIG. 3 is shown as an example. The techniques as set forth in this disclosure may be implemented in the example external programmer 300 of FIG. 3, as well as other types of systems not described specifically herein. Nothing in this disclosure should be construed so as to limit the techniques of this disclosure to the example architecture illustrated by FIG. 3.

FIG. 4 is a conceptual diagram illustrating an example user interface 400 configured to display a maximum selectable value of one or more electrical stimulation parameters. User interface 400 may be an example of user interface 306 of FIG. 3, which may include one or more displays.

In the example shown, user interface 400 includes graphical indicators 402, 404, and 406. Graphical indicators 402, e.g., 402a, 402b, and 402c (collectively “graphical indicators 402” or “vertebrae 402”)) graphically illustrate the T8, T9, and T10 vertebrae of a patient, e.g., patient 102. Graphical indicators 404, e.g., 404a and 404b (collectively “graphical indicators 404” or “leads 404”) graphically illustrate electrical stimulation leads, e.g., leads 130a and 130b, respectively. Graphical indicators 406a indicate electrodes 0-7 of lead 404a and graphical indicators 406b indicate electrodes 8-15 of lead 404b (collectively “graphical indicators 406” or “electrodes 406”). Vertebrae 402, leads 404, and electrodes 406 indicate the positions of the individual stimulation electrodes and leads in relation to patient anatomy, e.g., axial and lateral locations (relative to midline 418 of vertebrae 402) of leads 404. User interface 400 is configured to display an indicator, such as an icon, representing each of electrodes 0-15 along with labels “0” to “15” of each individual electrode. User interface also includes icons and labels for the individual leads 404 and vertebrae 402. In some examples, leads 404 and/or electrodes 406 may be displayed without vertebrae 402.

User interface 400 is configured to receive user input, e.g., via user interaction with user interface 400 such as a user selecting parameters, values, or options presentable in user interface 400 via selectable icons, menus, text boxes, widgets, and the like. User interface is also configured to indicate, e.g., visually to a user, various values and electrical stimulation therapy information, such as electrical stimulation parameter values. For example, electrodes 406 may indicate present (e.g., current or contemporary) values associated with one or more electrical stimulation parameters. In the example shown, an electrical stimulation program may specify that electrode combination of electrodes 3, 5, 9 and 11 at a total current output of 4.8 mA, with electrodes 3 and 9 defining first second cathodes 408a and 408b, respectively, and electrodes 5 and 11 defining first and second anodes 414a and 414b, respectively. The electrical stimulation program may specify that about 50% of the total current output of 4.8 mA is sunk by each of anodes 414a and 414b, e.g., discharged from each of cathodes 408a and 408b. User interface 400 includes output indicators 416a, 416b that are configured to indicate the relative amount of electrical current amplitude output by the present electrode combination. In the example shown, output indicators 416a, 416b indicate the relative amount of total output electrical current that is sunk to each of anodes 414a, 414b, e.g., about 50% by each of anodes 414a, 414b.

In some examples, user interface 400 may be configured to indicate the relative amount of the total output electrical current sourced by one or more electrodes of an electrode combination. For example, user interface 400 includes output indicator 410a configured to indicate the present electrical current amplitude sourced by cathode 408a (e.g., 2.5 mA in the example shown) and output indicator 412a configured to indicate the present maximum electrical current amplitude that could be sourced by cathode 408a (e.g., 7.9 mA in the example shown). User interface 400 also includes output indicator 410b configured to indicate the present electrical current amplitude sourced by cathode 408b (e.g., 2.3 mA in the example shown) and output indicator 412b configured to indicate the present maximum electrical current amplitude that could be sourced by cathode 408b (e.g., 8.4 mA in the example shown). In some examples, any or all of output indicators 410a, 410b, 412a, 412b, 416a, and 416b may be configured to indicate the amplitude output by the electrode combination as a percentage of the total output current and/or as electrical currents values (e.g., in mA) of one or more of the electrodes of the electrode combination.

In the example shown, indicators 410a, 410b, 412a, and 412b provide information to the user, clinician, or patient regarding a present value of one or more electrical stimulation parameter values (e.g., the electrical current output of a particular electrode combination in the example shown) relative to the maximum possible values for each active electrode, cathode or anode. In some examples, user interface 400 may be configured to indicate or display the relative values of the present and maximum possible electrical stimulation parameters of individual electrodes, combinations of electrodes, or sub-combinations of electrodes (e.g., anode-cathode pair 408a, 414a and anode-cathode pair 408b, 414b). These values for maximum possible values may be displayed next to or adjacent the respective electrodes as shown in the example of FIG. 4, or provided elsewhere on the user interface, such as in a table or in a pop-up window that is presented in response to user selection of the electrode (e.g., clicking, hovering over, or otherwise receiving user request to view the electrode information).

User interface 400 includes output indicator 420 configured to indicate the present total electrical current amplitude of the entire electrode combination (e.g., 4.8 mA in the example shown), output indicator 422 configured to indicate the present maximum electrical current amplitude that entire electrode combination could output (e.g., 16.5 mA in the example shown), and control indicator 424 configured to graphically indicate the present total electrical current amplitude of the entire electrode combination relative to the present maximum electrical current amplitude that entire electrode combination could output, e.g., as a radial slider knob at a particular position along a radial path 426 from minimum to maximum, and configured to allow a user to control the present total electrical current amplitude of the entire electrode combination, e.g., via manipulating/sliding the knob along the radial path. In this manner, the maximum electrical current amplitude is an example of the maximum selectable value for amplitude in the example of FIG. 4. In the example shown, indicators 420, 422, and 424 provide information to the user, clinician, or patient regarding a present value of one or more electrical stimulation parameter value (e.g., the electrical current output of an entire electrode combination in the example shown) relative to the maximum selectable value.

Although shown as a radial slider knob 424, output indicator 422 as text, and output indicator 420 as text in a dropdown box or menu, the information displayed and/or output by indicators 420-426 may be in other formats. For example, user interface 400 may be configured to output a plot, heat map, topographical map, or the like, configured to indicate a maximum selectable value of one or more electrical stimulation parameters relative to one or more electrical characteristics of the stimulation system and/or one or more other electrical stimulation parameters.

For example, user interface 400 may be configured to output a plot (e.g., a 2D plot) in which the y-axis indicates the maximum selectable value of a first electrical stimulation parameter (e.g., electrical current or voltage amplitude) as a function of the value of a second electrical stimulation parameter (e.g., pulse frequency, pulse width, an electrode combination or a number of cathodes or anodes) plotted on the x-axis. In some examples, user interface 400 may be configured to output a topographical map (e.g., a 3D plot) in which one of the axes, such as the z-axis, indicates the maximum selectable value of a first electrical stimulation parameter (e.g., electrical current or voltage amplitude) as a function of the values of two other electrical stimulation parameters (e.g., pulse frequency, pulse width, an electrode combination or a number of cathodes or anodes) plotted on the x-axis and y-axis. In some examples, user interface 400 may be configured to output a 3D plot configured to indicate maximum selectable values for a plurality of electrical stimulation parameters (e.g., two or three in the case of a 3D plot), e.g., a “volume” indicating the selectable values of three electrical stimulation parameters plotted on the x-, y-, and z-axes, with the surface of the volume indicating the maximum and/or minimum selectable values of each of the three electrical stimulation parameters in relation to each other. In some examples, user interface 400 may be configured to output a 2D plot or image, such as a heat map, in which color indicates the maximum selectable value of a first electrical stimulation parameter (e.g., electrical current or voltage amplitude) as a function of the values of two other electrical stimulation parameters (e.g., pulse frequency, pulse width, an electrode combination or a number of cathodes or anodes) plotted on the x-axis and y-axis. In some examples, user interface 400 is configured to indicate one or more combinations of selectable electrical stimulation parameters are achievable with given, or determined, electrical characteristics (e.g., impedance of one or more electrodes, electrical efficiency of one or more electrodes, charge density, pulse width, frequency, battery power, battery capacity, battery life, device life, recharge burden, or the like).

In some examples, one or more of indicators 410a, 410b, 412a, 412b, 414a, 414b, 416a, 416b, 420, 422, or any other indicator of user interface 400 may be user-selectable and configured to receive user input changing the relative amount of the electrical stimulation parameter, e.g., electrical current amplitude in the example shown, that is to be output to the electrode combination, e.g., the anodes and cathodes 408a, 414a, 408b, 414b in the example shown. In other words, output indicators 410a, 410b, 412a, 412b, 414a, 414b, 416a, 416b, 420, 422, or any other indicator of user interface 400, may be more than simple indicators and may be configured to receive user input as well.

In some examples, the maximum selectable value of the entire electrode combination indicated by indicator 422 may not equal the sum of the maximum selectable values of individual electrodes or electrode sub-combinations. In the example shown, the maximum selectable values indicated by indicators 412a and 412b sum to 16.3 mA, whereas the maximum selectable value for the particular electrical stimulation parameter for that set of stimulation parameters, e.g., electrical current amplitude, is higher at 16.5 mA. In some examples, such a difference may indicate that the current electrode combination, e.g., electrodes 3, 5, 9 and 11, do not output the maximum possible electrical current amplitude, for example, because of the impedances and/or efficiencies of that particular electrode combination. In some examples, such a difference may indicate that IMD 110 may be able to output more electrical current amplitude for a different combination of electrical stimulation parameter values, e.g., different pulse widths, frequencies, numbers of electrodes, or the like.

In this way, user interface 400 is configured to display granular information to the user regarding where the present electrical stimulation parameter values are at relative to their possible maximum values, thereby improving the user's knowledge and ability to tailor the present electrical stimulation therapy to the present needs of the patient and in anticipation of future electrical stimulation therapy needs of the patient. In other words, user interface 400 is configured to present information (e.g., maximum possible electrical stimulation parameters) to allow the user to tailor electrical stimulation programs to leave “head room” for future changes. For example, without such information, the user may program IMD 110 with electrical stimulation parameter values that give optimum present benefits and/or results to patient 102, but which may be at or near their respective maximum. With such information, the user may program IMD 110 with electrical stimulation parameter values that give good present benefits and/or results to patient 102 counter-balanced by the ability of IMD 110 to output different electrical stimulation parameter values should the needs of patient 102 change. In other words, with such information, the user may choose present electrical stimulation parameter values that do not give the present optimum benefits and/or results, but that have the ability to adjust to give optimum benefits and/or results over a period of time that includes changes to the electrical stimulation parameter values that patient 102 may need.

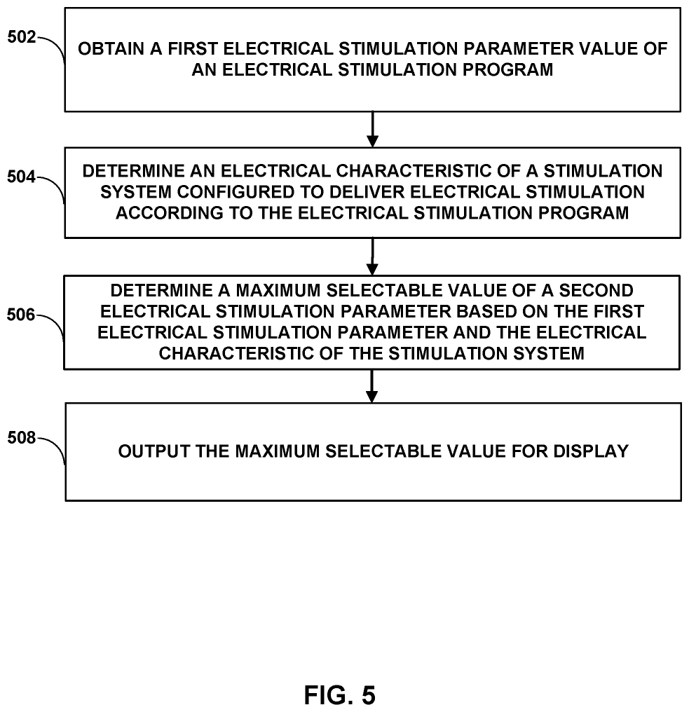

FIG. 5 is a flow diagram illustrating an example method for indicating a maximum selectable value of an electrical stimulation parameter within a user interface, in accordance with one or more techniques of this disclosure. FIG. 5 is described with respect to IMD 110, external programmer 104, and user interfaces 306 and 400. However, the example technique of FIG. 5 may be used with other electrical stimulation systems.

IMD 110 may obtain a first electrical stimulation parameter of an electrical stimulation program that defines an electrical stimulation therapy deliverable to a patient (502). For example, a clinician (or patient, or user) may select an electrical stimulation program using external programmer 104. External programmer 104 may output an indication of the selected program to IMD 110 and processing circuitry 208 may retrieve the electrical stimulation parameters defined by the program from memory 216, or processing circuitry 208 may receive the electrical stimulation parameters defined by the program from external programmer 104. In some examples, the first electrical stimulation parameter may be a number of electrodes, a number of anodes, a number of cathodes, a particular combination of electrodes, anodes, and/or cathodes, an electrical voltage and/or electrical current amplitude, a pulse width, or a pulse frequency.

IMD 110 may then determine an electrical characteristic of a stimulation system configured to deliver the electrical stimulation therapy according to the electrical stimulation program (504). For example, processing circuitry 208 may determine an impedance of each of the electrodes, anodes, and/or cathodes of the electrical stimulation program based on respective signals sent through the different combinations of electrodes. In some examples, processing circuitry 208 may determine an efficiency of an electrode, anode, and/or cathode, e.g., based on an electrical characteristic of the of the stimulation system. For example, processing circuitry 208 may determine an efficiency of each anode and/or cathode, e.g., 408a, 414a and 408b, 414b, based on an impedance of each anode and/or cathode 408a, 414a and 408b, 414b. In some examples, processing circuitry 208 may determine an amount of battery power available for the electrical stimulation program and/or a rate of use of battery power by the electrical stimulation program. In some examples, processing circuitry 208 may determine one or more electrical characteristics of the stimulation system configured to deliver the electrical stimulation therapy according to a plurality of electrodes, anodes, and/or cathodes, e.g., an electrode combination and/or sub-combination.

IMD 110 may determine a maximum selectable value for a second electrical stimulation parameter of the electrical stimulation program based on the electrical characteristic and the at least one first electrical stimulation parameter (506). In some examples, the second electrical stimulation parameter is at least one of an electrical current amplitude or a voltage amplitude. For example, processing circuitry 208 may determine a maximum selectable electrical current amplitude value based on an impedance of one or more electrode and at least one of the number of electrodes, the pulse width, or the pulse frequency of the electrical stimulation program.

IMD 110 may output the maximum selectable value for the second electrical stimulation parameter of the electrical stimulation program for display by a user interface (508). For example, processing circuitry 208 may output the maximum selectable value to user interface 400 for display, e.g., via indicators 410a, 410b, 412a, 412b, 414a, 414b, 416a, 416b, 420, 422, or any other indicator of user interface 400.

In some examples, processing circuitry 208 may determine that the presently selected value of an electrical stimulation parameter is greater than the maximum selectable value. Responsive to such a determination, processing circuitry 208 may output an alert indicating that the presently selected value is greater than the maximum selectable value for display by user interface 400. For example, if a user selects output indicator 420 of user interface 400, corresponding to the total electrical presently amplitude of the electrical stimulation therapy, and selects and/or enters a value greater than the maximum selectable value, e.g., the user inputs 17 mA when the maximum selectable value is 16.5 mA as shown, processing circuitry 208 may output an alert, e.g., visual, audible, tactile such as a vibration, an electrical alert signal to a different device, or the like. In other examples, processing circuitry 208 may prevent the user from selecting a value greater than the maximum selectable value.

In some examples, processing circuitry 208 may determine a headroom amount that is the difference between the maximum selectable value and the presently selected value. For examples, if the presently selected value of the total electrical current is 4.8 mA (e.g., indicated by indicator 420) and the maximum selectable value is 16.5 mA (e.g., indicated by indicator 422), processing circuitry 208 may determine a 11.7 mA headroom for the total electrical current amplitude parameter. In some examples, based on there being headroom, e.g., the presently selected value being less than or equal to than the maximum selectable value, processing circuitry 208 may output the headroom amount and/or value (e.g., the 11.7 mA in the example above) for display, e.g., by user interface 400. In some examples, processing circuitry 208 may determine a headroom amount for each electrode 132 (indicated as electrodes 406a, 406b in FIG. 4), and/or each anode or cathode of the electrical stimulation program, and output the headroom amount for each electrode, anode, or cathode for display, e.g., by user interface 400. For example, processing circuitry 208 may determine a headroom amount of 5.4 mA for anode 408a and/or cathode 414a and determine a headroom amount of 6.1 mA for anode 408b and/or cathode 414b, and output each headroom amount for display by user interface 400 (not shown).

In some examples, processing circuitry 208 may determine an electrical stimulation program for which the maximum selectable value of an electrical stimulation parameter is the greatest. In other words, processing circuitry 208 may determine a plurality of maximum selectable values of a particular electrical stimulation parameter each corresponding to an electrical stimulation program of a plurality of electrical stimulation programs, e.g., sets of electrical stimulation parameters, and determine which electrical stimulation program yields the greatest headroom for that particular electrical stimulation parameter. For example, processing circuitry 208 may determine a maximum selectable value for an electrical current amplitude for each of a plurality of programs varying the electrode combination and/or number of electrodes, pulse frequency, and/or pulse width electrical stimulation parameters, and determine which combination/program results in the greatest maximum selectable value. In some examples, processing circuitry 208 may determine such a greatest maximum selectable value also based on at least one electrical characteristic of the stimulation system, e.g., an impedance, battery power, electrodes efficiencies, or the like. Processing circuitry 208 may output, for display by user interface 400, the one or more parameter values of the electrical stimulation program yielding the greatest maximum selectable value of the particular electrical stimulation parameter and/or the corresponding headroom for that particular electrical stimulation parameter and electrical stimulation program.

In some examples, processing circuitry 208 may determine a threshold value of an electrical stimulation parameter at which respective evoked compound action potential (ECAP) signals are detectable and output, for display by user interface 400, an indication of a relationship between the threshold value and the maximum selectable value of that electrical stimulation parameter. For example, processing circuitry 208 may determine a threshold value of an electrical current amplitude and output for display by user interface 400 a relationship between the threshold value and the maximum selectable electrical current amplitude, e.g., a difference between the maximum selectable electrical current amplitude value and the value at which an ECAP is detectable by leads 130. In some examples, processing circuitry 208 may determine a threshold amplitude value (e.g., electrical current or voltage amplitude) at which an ECAP is detectable for a plurality of electrode stimulation programs and/or electrode combinations, and output the results for display by user interface 400.

The techniques of this disclosure may also be described in the following examples.

Example 1: A method includes: obtaining, by processing circuitry, at least one first electrical stimulation parameter of an electrical stimulation program that defines an electrical stimulation therapy deliverable to a patient, determining, by processing circuitry, an electrical characteristic of a stimulation system configured to deliver the electrical stimulation therapy according to the electrical stimulation program; determining, by processing circuitry and based on the electrical characteristic and the at least one first electrical stimulation parameter, a maximum selectable value for a second electrical stimulation parameter of the electrical stimulation program; and outputting, for display by a user interface, the maximum selectable value.

Example 2: The method of example 1, wherein the at least one first electrical stimulation parameter comprises a number of anodes, a number of cathodes, a pulse width, or a pulse frequency.

Example 3: The method of example 2, wherein the electrical characteristic comprises an impedance of each of the anodes and cathodes of the electrical stimulation program.

Example 4: The method of any one of examples 1 through 3, wherein the second electrical stimulation parameter is at least one of an electrical current amplitude or a voltage amplitude.

Example 5: The method of any one of examples 1 through 4, further includes determining, based on the maximum selectable value and the presently selected value for the second electrical parameter, that the presently selected value is greater than the maximum selectable value; and responsive to determining that the presently selected value is greater than the maximum selectable value, outputting, for display by the user interface, an alert indicating that the presently selected value is greater than the maximum selectable value.

Example 6: The method of any one of examples 1 through 5, further includes determining, based on the maximum selectable value and the presently selected value for the second electrical parameter, a headroom amount that is the difference between the maximum selectable value and the presently selected value, wherein the presently selected value is less than or equal to the maximum selectable value; and outputting, for display by the user interface and based on the presently selected value being less than or equal to than the maximum selectable value, the headroom amount.

Example 7: The method of example 6, wherein the headroom amount includes a headroom amount for each anode and cathode of the electrical stimulation program, and wherein the method further comprises outputting, for display by the user interface, the headroom amount for each anode and cathode.

Example 8: The method of any one of examples 1 through 7, further includes determining, by processing circuitry and based on the electrical characteristic, an efficiency of an anode and a cathode of the stimulation system; and outputting, for display by the user interface, the efficiency of the anode and cathode.

Example 9: The method of any one of examples 1 through 8, further includes determining, an electrical characteristic of a stimulation system configured to deliver the electrical stimulation therapy via a plurality of anodes and cathodes; determining, based on the electrical characteristic, an electrical stimulation program for which the maximum selectable value for the second electrical stimulation parameter is the greatest, wherein the at least one first electrical stimulation parameter comprises at least one of a combination of anodes and cathodes, a pulse width, or a pulse frequency; and outputting, for display by the user interface, one or more parameter values of the electrical stimulation program.

Example 10: The method of any one of examples 1 through 9, further includes determining, for the electrical stimulation program, an amplitude threshold at which respective evoked compound action potential (ECAP) signals are detectable; and outputting, for display by the user interface, an indication of a relationship between the amplitude threshold and the maximum selectable value.

Example 11: A system includes: processing circuitry configured to: obtain a first electrical stimulation parameter of an electrical stimulation program that defines an electrical stimulation therapy deliverable to a patient, determine an electrical characteristic of a stimulation system configured to deliver the electrical stimulation therapy according to the electrical stimulation program; determine, based on the electrical characteristic and the at least one first electrical stimulation parameter, a maximum selectable value for a second electrical stimulation parameter of the electrical stimulation program; and output, for display by a user interface, the maximum selectable value.

Example 12: The system of example 11, wherein the first electrical stimulation parameter comprises a number of anodes, a number of cathodes, a pulse width, or a pulse frequency.

Example 13: The system of any of examples 11 and 12 ore any of examples 11 and 12, wherein the electrical characteristic comprises an impedance of each of the anodes and cathodes of the electrical stimulation program.

Example 14: The system of any one of examples 11 through 13, wherein the second electrical stimulation parameter is at least one of an electrical current amplitude or a voltage amplitude.

Example 15: The system of any one of examples 11 through 14, wherein the processing circuitry is further configured to: determine, based on the maximum selectable value and the presently selected value for the second electrical parameter, that the presently selected value is greater than the maximum selectable value; and responsive to determining that the presently selected value is greater than the maximum selectable value, output, for display by the user interface, an alert indicating that the presently selected value is greater than the maximum selectable value.

Example 16: The system of any one of examples 11 through 15, wherein the processing circuitry is further configured to: determine, based on the maximum selectable value and the presently selected value for the second electrical parameter, a headroom amount that is the difference between the maximum selectable value and the presently selected value, wherein the presently selected value is less than or equal to the maximum selectable value; and output, for display by the user interface and based on the presently selected value being greater than the maximum selectable value, the headroom amount.

Example 17: The system of example 16, wherein the headroom amount includes a headroom amount for each anode and cathode of the electrical stimulation program, and wherein the method further comprises outputting, for display by the user interface, the headroom amount for each anode and cathode.

Example 18: The system of any one of examples 11 through 17, wherein the processing circuitry is further configured to: determine, based on the electrical characteristic, an efficiency of an anode and a cathode of the stimulation system; and output, for display by a user interface, the efficiency of the anode and cathode.

Example 19: The system of any of examples 11 through 18, further comprising an external programmer comprising the processing circuitry and the user interface.

Example 20: A non-transitory computer-readable medium comprising instructions that, when executed, control processing circuitry to: obtain a first electrical stimulation parameter of an electrical stimulation program that defines an electrical stimulation therapy deliverable to a patient, determine an electrical characteristic of a stimulation system configured to deliver the electrical stimulation therapy according to the electrical stimulation program; determine, based on the electrical characteristic and the at least one first electrical stimulation parameter, a maximum selectable value for a second electrical stimulation parameter of the electrical stimulation program; and output, for display by a user interface, the maximum selectable value.

Example 21: The non-transitory computer-readable medium of example 20, further comprising instructions that cause the processing circuitry to: determine, based on the maximum selectable value and the presently selected value for the second electrical parameter, that the presently selected value is greater than the maximum selectable value; and responsive to determining that the presently selected value is greater than the maximum selectable value, output, for display by the user interface, an alert indicating that the presently selected value is greater than the maximum selectable value.

The techniques described in this disclosure may be implemented, at least in part, in hardware, software, firmware or any combination thereof. For example, various aspects of the described techniques may be implemented within one or more processors or processing circuitry, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit including hardware may also perform one or more of the techniques of this disclosure.

Such hardware, software, and firmware may be implemented within the same device or within separate devices to support the various operations and functions described in this disclosure. In addition, any of the described units, circuits or components may be implemented together or separately as discrete but interoperable logic devices. Depiction of different features as circuits or units is intended to highlight different functional aspects and does not necessarily imply that such circuits or units must be realized by separate hardware or software components. Rather, functionality associated with one or more circuits or units may be performed by separate hardware or software components or integrated within common or separate hardware or software components.

The techniques described in this disclosure may also be embodied or encoded in a computer-readable medium, such as a computer-readable storage medium, containing instructions that may be described as non-transitory media. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

What is claimed is:

1. A method comprising:
obtaining, by processing circuitry, at least one first electrical stimulation parameter of an electrical stimulation program that defines an electrical stimulation therapy deliverable to a patient,
determining, by processing circuitry, an electrical characteristic of a stimulation system configured to deliver the electrical stimulation therapy according to the electrical stimulation program;
determining, by processing circuitry and based on the electrical characteristic and the at least one first electrical stimulation parameter, a maximum selectable value for a second electrical stimulation parameter of the electrical stimulation program; and
outputting, for display by a user interface, the maximum selectable value.

2. The method of claim 1, wherein the at least one first electrical stimulation parameter comprises a number of anodes, a number of cathodes, a pulse width, or a pulse frequency.

3. The method of claim 2, wherein the electrical characteristic comprises an impedance of each of the anodes and cathodes of the electrical stimulation program.

4. The method of claim 1, wherein the second electrical stimulation parameter is at least one of an electrical current amplitude or a voltage amplitude.

5. The method of claim 1, further comprising:
determining, based on the maximum selectable value and the presently selected value for the second electrical parameter, that the presently selected value is greater than the maximum selectable value; and
responsive to determining that the presently selected value is greater than the maximum selectable value, outputting, for display by the user interface, an alert indicating that the presently selected value is greater than the maximum selectable value.

6. The method of claim 1, further comprising:
determining, based on the maximum selectable value and the presently selected value for the second electrical parameter, a headroom amount that is the difference between the maximum selectable value and the presently selected value, wherein the presently selected value is less than or equal to the maximum selectable value; and
outputting, for display by the user interface and based on the presently selected value being less than or equal to than the maximum selectable value, the headroom amount.

7. The method of claim 6, wherein the headroom amount includes a headroom amount for each anode and cathode of the electrical stimulation program, and wherein the method further comprises outputting, for display by the user interface, the headroom amount for each anode and cathode.

8. The method of claim 1, further comprising:
determining, by processing circuitry and based on the electrical characteristic, an efficiency of an anode and a cathode of the stimulation system; and
outputting, for display by the user interface, the efficiency of the anode and cathode.

9. The method of claim 1, further comprising:
determining, an electrical characteristic of a stimulation system configured to deliver the electrical stimulation therapy via a plurality of anodes and cathodes;
determining, based on the electrical characteristic, an electrical stimulation program for which the maximum selectable value for the second electrical stimulation parameter is the greatest, wherein the at least one first electrical stimulation parameter comprises at least one of a combination of anodes and cathodes, a pulse width, or a pulse frequency; and
outputting, for display by the user interface, one or more parameter values of the electrical stimulation program.

10. The method of claim 1, further comprising:
determining, for the electrical stimulation program, an amplitude threshold at which respective evoked compound action potential (ECAP) signals are detectable; and
outputting, for display by the user interface, an indication of a relationship between the amplitude threshold and the maximum selectable value.

11. A system comprising:
processing circuitry configured to:
obtain a first electrical stimulation parameter of an electrical stimulation program that defines an electrical stimulation therapy deliverable to a patient,
determine an electrical characteristic of a stimulation system configured to deliver the electrical stimulation therapy according to the electrical stimulation program;
determine, based on the electrical characteristic and the at least one first electrical stimulation parameter, a maximum selectable value for a second electrical stimulation parameter of the electrical stimulation program; and
output, for display by a user interface, the maximum selectable value.

12. The system of claim 11, wherein the first electrical stimulation parameter comprises a number of anodes, a number of cathodes, a pulse width, or a pulse frequency.

13. The system of claim 11, wherein the electrical characteristic comprises an impedance of each of the anodes and cathodes of the electrical stimulation program.

14. The system of claim 11, wherein the second electrical stimulation parameter is at least one of an electrical current amplitude or a voltage amplitude.

15. The system of claim 11, wherein the processing circuitry is further configured to:
determine, based on the maximum selectable value and the presently selected value for the second electrical parameter, that the presently selected value is greater than the maximum selectable value; and
responsive to determining that the presently selected value is greater than the maximum selectable value, output, for display by the user interface, an alert indicating that the presently selected value is greater than the maximum selectable value.

16. The system of claim 11, wherein the processing circuitry is further configured to:
determine, based on the maximum selectable value and the presently selected value for the second electrical parameter, a headroom amount that is the difference between the maximum selectable value and the presently selected value, wherein the presently selected value is less than or equal to the maximum selectable value; and
output, for display by the user interface and based on the presently selected value being greater than the maximum selectable value, the headroom amount.

17. The system of claim 16, wherein the headroom amount includes a headroom amount for each anode and cathode of the electrical stimulation program, and wherein the method further comprises outputting, for display by the user interface, the headroom amount for each anode and cathode.

18. The system of claim 11, wherein the processing circuitry is further configured to:
determine, based on the electrical characteristic, an efficiency of an anode and a cathode of the stimulation system; and
output, for display by a user interface, the efficiency of the anode and cathode.

19. The system of claim 11, further comprising an external programmer comprising the processing circuitry and the user interface.

20. A non-transitory computer-readable medium comprising instructions that, when executed, control processing circuitry to:
obtain a first electrical stimulation parameter of an electrical stimulation program that defines an electrical stimulation therapy deliverable to a patient,
determine an electrical characteristic of a stimulation system configured to deliver the electrical stimulation therapy according to the electrical stimulation program;
determine, based on the electrical characteristic and the at least one first electrical stimulation parameter, a maximum selectable value for a second electrical stimulation parameter of the electrical stimulation program; and
output, for display by a user interface, the maximum selectable value.
